(19) United States
(12) Patent Application Publication
(10) Pub. No.: US 2010/0028420 A1
(43) Pub. Date: Feb. 4, 2010

(54) CONTROLLED RELEASE COMPOSITION AND PROCESS

(75) Inventor: L. Charles Hardy, St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company, Saint Paul, MN (US)

(21) Appl. No.: 12/519,258

(22) PCT Filed: Dec. 18, 2007
(86) PCT No.: PCT/US2007/087867

§ 371 Date: Jun. 15, 2009

Related U.S. Application Data
(60) Provisional application No. 60/871,530, filed on Dec. 22, 2006.

(51) Int. Cl. A61K 9/48; A61K 9/20; A61K 9/14; A61K 38/00

(52) U.S. Cl. 424/451; 424/464; 424/484; 514/2

(57) ABSTRACT

A composition for encapsulation and controlled release comprises a water-insoluble matrix comprising a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety. The composition can further comprise a guest molecule (for example, a drug) that can be encapsulated within the matrix and subsequently released.

### STATEMENT OF PRIORITY

This application claims the priority of U.S. Provisional Application No. 60/871,530 filed Dec. 22, 2006, the contents of which are hereby incorporated by reference.

### FIELD

This invention relates to compositions and processes useful for the encapsulation and controlled release of guest molecules (for example, drugs).

### BACKGROUND

Controlled release compositions and methods have found broad utility and have been particularly useful in the field of drug delivery. Controlled release has been achieved by a number of different methods.

For example, polymers have been used to surround or to form a mixture with a substance and to control release of the substance through swelling of the polymer in the presence of water. This approach has relied upon the mechanism of diffusion of the substance through a swollen polymer matrix. Other polymer-based approaches have relied upon polymer erosion or degradation to control release. Since most polymers are highly polydisperse in nature, however, the release rate in polymer systems can be difficult to control. In addition, there are only a limited number of polymers that are suitable for use in pharmaceutical applications, and a given polymer can interact with various different substances in quite different and unpredictable ways.

Another common approach has been to use macroscopic structures having openings or membranes that allow for the release of a substance. Macroscopic structures, such as osmotic pumps, have been used to control release by uptake of water from the environment into a chamber containing a substance that can be forced from the chamber through a delivery orifice. This has required the preparation of complex structures and the filling of such structures with the substance to be delivered.

Protection of a drug from adverse environmental conditions can be desirable in certain drug delivery applications. The human gastrointestinal tract is one example of an environment that can interfere with the therapeutic efficacy of a drug. Thus, the ability to selectively protect a drug from certain environmental conditions, such as the low pH of the stomach, and to also be able to selectively and controllably deliver the drug under other environmental conditions, such as the neutral pH of the small intestine, is highly desirable.

Alteration and control of the rate at which a drug is released to a bioactive receptor (that is, sustained or controlled drug release) can also be desirable in certain drug delivery applications. Sustained or controlled drug release can have the desirable effects of reducing dosing frequency, reducing side effects, and increasing patient compliance.

### SUMMARY

Thus, we recognize that there is a need for industrially useful compositions and processes for effectively and efficiently controlling the release of various substances, including drugs (particularly pH-sensitive drugs). In particular, we recognize that there is a need for compositions and processes for orally delivering insulin to diabetics, so as to reduce or eliminate the need for insulin delivery by injection.

Briefly, in one aspect, this invention provides a composition for encapsulation and controlled release comprising a water-insoluble matrix comprising a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety. The composition can further comprise a guest molecule (for example, a drug) that can be encapsulated within the matrix and subsequently released.

Preferably, the host molecule comprises at least one pteroyl or 5-substituted pteroyl moiety. More preferably, the host molecule is a pteroylglutamic acid (for example, folic acid) or a 5-substituted pteroylglutamic acid (for example, folinic acid).

It has been discovered that host molecules having certain above-described structural characteristics can exhibit, upon base addition, unexpected neutralization behavior in the form of self-buffering characteristics. Such characteristics enable the formation of a liquid crystalline state (for example, a chromonic phase) without significant variations in pH. This makes the host molecules especially well-suited for the encapsulation and delivery of pH-sensitive drugs (for example, oral delivery of proteinaceous drugs such as insulin).

In addition, the neutralized host molecules can exhibit a broad liquid crystal range (for example, over a range of about 1 equivalent to about 2 equivalents of added base). This facilitates their use in the formation of a water-insoluble matrix and/or crosslinked particles or beads (for example, by the addition of multi-valent cations) and further makes them well-suited for use in robust industrial processes (for example, automated processing). The liquid crystalline behavior of partially neutralized folic acid, in particular, is surprising in view of its reported insolubility in the requisite pH range.

In another aspect, this invention provides a particulate composition comprising particles comprising a water-insoluble matrix comprising a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety. The particulate composition can further comprise a guest molecule (for example, a drug) that can be encapsulated within the matrix and subsequently released.

In yet another aspect, this invention also provides a medicinal suspension formulation comprising the particulate composition of the invention and at least one liquid (for example, at least one liquid, pharmaceutically acceptable carrier).

In other aspects, this invention provides a tablet comprising the composition of the invention and a capsule comprising the particulate composition of the invention (both the tablet and the capsule optionally further comprising at least one pharmaceutically acceptable carrier).

In a further aspect, this invention provides a process for preparing the composition of the invention. The process comprises:
(a) combining a dispersion (preferably, a dispersion in water or in a mixture of water and organic solvent) comprising at least one of the above-described host molecules and at least one base to form a solution having a chromonic phase; and
(b) combining the solution having a chromonic phase with a solution of multi-valent cations to form a water-insoluble matrix.

In yet another aspect, this invention provides a process for drug delivery, which comprises:
(a) providing the composition of the invention comprising a water-insoluble matrix and at least one drug encapsulated within the matrix;
(b) delivering the composition to an organism such that it comes into contact with a composition comprising univalent cations and releases at least a portion of the encapsulated drug; and
(c) allowing the released drug to remain in contact with at least a part of the organism for a period of time sufficient to achieve a desired therapeutic effect.

### BRIEF DESCRIPTION OF DRAWINGS

These and other features, aspects, and advantages of the present invention will become better understood with regard to the following description, appended claims, and accompanying drawing, wherein:

FIGS. 1a and 1b are schematic representations of embodiments of an individual host molecule or association of host molecules (for example, a lateral association) and an individual multi-valent cation, respectively.

FIG. 2 is a schematic representation of an embodiment of a water-insoluble matrix.

FIG. 3 is a schematic representation of an embodiment of a water-insoluble matrix comprising an encapsulated guest molecule.

FIG. 4 is a schematic representation of dissociation of the components of an embodiment of a water-insoluble matrix and release of its guest molecule in the presence of univalent cations.

FIG. 5 is a titration curve (plot of pH versus milliliters of 0.5 weight percent base) for the titration of a dispersed solid comparative chromonic compound described in the “Comparative Titration” of the Examples section below.

FIG. 6 is a titration curve (plot of pH versus milliliters of 1.0 weight percent base) for the titration of dispersed solid folic acid described in “Titration A” of the Examples section below.

FIG. 7 is a titration curve (plot of pH versus milliliters of 0.5 weight percent base) for the titration of dispersed solid folic acid described in “Titration B” of the Examples section below.

### DETAILED DESCRIPTION

As summarized above, this invention provides a composition for encapsulation and controlled release comprising a water-insoluble matrix. The water-insoluble matrix comprises a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety.

Preferably, the host molecule comprises at least one pteroyl or 5-substituted pteroyl moiety. More preferably, the host molecule is a pteroylglutamic acid (for example, folic acid) or a 5-substituted pteroylglutamic acid (for example, folinic acid).

The composition can further comprise a guest molecule (for example, a drug) that can be encapsulated within the matrix and subsequently released. In at least some embodiments of the composition, the matrix can selectively protect a drug from certain environmental conditions and then controllably deliver the drug under other environmental conditions. For example, the matrix can be stable in the acidic environment of an animal's stomach and then dissolve when passed into the non-acidic environment of the animal's intestine, and the matrix can be used to protect a drug from enzymatic degradation.

Various embodiments of the composition comprise matrices that can effectively isolate drug molecules in a particle, such that unfavorable interactions (for example, chemical reactions) between different drugs in a combination dosage form, unfavorable changes in a single drug component (for example, Ostwald ripening or particle growth and changes in crystalline form), and/or unfavorable interactions between a drug and one or more excipients can be avoided. The matrix can allow two drugs (or a drug and an excipient) that are ordinarily unstable in each other's presence to be formulated into a stable dosage form.

### Chemical Structures

As used in this patent application:

“folic acid” means N-[4-[[(2-amino-1,4-dihydro-4-oxo-6-pteridinyl)methyl]amino]benzoyl-L-glutamic acid, which can be represented by the structural formula

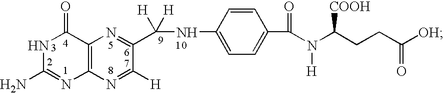

“folinic acid” means N-[4-[[(2-amino-5-formyl-1,4,5,6,7,8-hexahydro-4-oxo-6-pteridinyl)methyl]amino]benzoyl]-L-glutamic acid, which can be represented by the structural formula

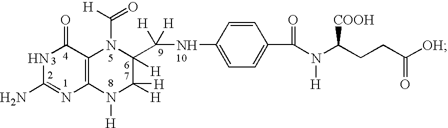

“non-covalent” (in reference to a crosslinking bond) means that the crosslinking bond can be formed and cleaved reversibly in the presence of a solvent;

“non-interfering” (in reference to a substituent on a host molecule) means that the substituent is of a size and chemical nature such that it does not prevent an at least partially neutralized host molecule from forming a liquid crystalline phase when dispersed in a liquid medium;

“organic group” means a hydrocarbyl group or a hydrocarbyl group that contains at least one heteroatom (for example, oxygen, nitrogen, halogen, and/or sulfur);

“pterin moiety” means the monovalent moiety that is represented by the structural formula

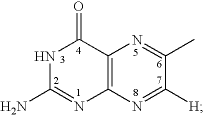

“5-substituted pterin moiety” means the monovalent moiety that is represented by the structural formula below (where R is a non-interfering organic group)

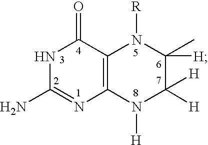

“pteroyl moiety” means the monovalent moiety that is represented by the structural formula

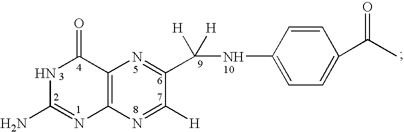

“5-substituted pteroyl moiety” means the monovalent moiety that is represented by the structural formula below (where R is a non-interfering organic group)

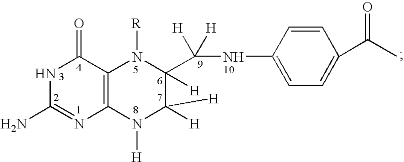

“pteroylglutamic acid” means an acid (or a mixture of acids) that is represented by the structural formula below (where n is an integer of at least 1, preferably from 1 to about 7)

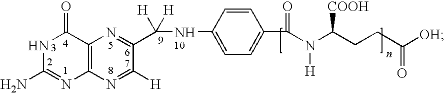

“5-substituted pteroylglutamic acid” means an acid (or a mixture of acids) that is represented by the structural formula below (where R is a non-interfering organic group; and n is an integer of at least 1, preferably from 1 to about 7)

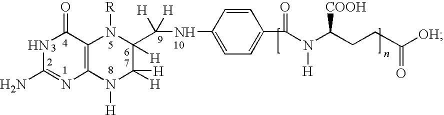

### Host Molecules

Suitable host molecules for use in the composition of the invention include those that can be non-covalently crosslinked by multi-valent cations, that are non-polymeric, that have more than one carboxy functional group, that have at least partial aromatic or heteroaromatic character, and that comprise at least one pterin or 5-substituted pterin moiety (more preferably, at least one pteroyl or 5-substituted pteroyl moiety). The substituent at the number 5 position of the moiety can be a non-interfering organic group. Preferred substituents include hydrogen, alkyl, formyl (HC(═O)—), formimino (HC(═NH)—), and multivalent “bridging” substituents (for example, alkylidene (—CHR—) and alkylidyne (—CR⁺—), where R is alkyl or hydrogen) that can be bonded to another atom of the host molecule (for example, replacing the hydrogen atom at the number 10 position of a pteroyl moiety) so as to form an alicyclic ring structure. More preferred substituents include hydrogen, alkyl, formyl, formimino, methylidene (or methylene, —CH₂—), and methylidyne (—CH⁺—) (even more preferably, hydrogen, alkyl, and formyl; most preferably, formyl). The substituents preferably have from 1 to about 12 non-hydrogen atoms (more preferably, from 1 to about 8; most preferably, from 1 to about 4).

As used herein, the term “non-polymeric” means that the host molecules are typically of relatively low molecular weight when compared to typical high molecular weight polymers (preferably having a molecular weight less than 2000 g/mol, more preferably less than 1000 g/mol, and most preferably less than 600 g/mol). Thus, non-polymeric host molecules include short chain oligomers having a small number of repeat units (for example, dimers, trimers, tetramers, and so forth, up to at least about 7 or 8 repeat units) and molecules that consist of a single unit (that is, not comprising repeat units).

Useful host molecules generally have more than one carboxy functional group, represented in its unionized form by the chemical structure —COOH. The host molecule can have several carboxy functional groups (for example, three carboxy functional groups), and preferably two carboxy functional groups. The carboxy groups can be attached to adjacent carbon atoms on the host molecule (that is, HOOC—C—C—COOH), but are usually attached to carbon atoms that are separated by one or more intervening atoms. As used herein, the term “carboxy functional group” is intended to encompass free ionized forms, such as —COO⁻, as well as salts of carboxy functional groups (that is, carboxylates), including, for example, sodium, potassium, and ammonium salts.

Useful host molecules generally have at least partial aromatic or heteroaromatic character. This means that at least one portion of the host molecule is characterized by a cyclic delocalized π-electron system. In general, these compounds all share the common characteristic of having delocalized π-electrons that can be expressed by using multiple resonance structures with 4n+2π-electrons. The term “aromatic” refers to ring structures containing only carbon (examples include phenyl and naphthyl groups), and the term “heteroaromatic” refers to ring structures that contain at least one atom other than carbon (for example, nitrogen, sulfur, or oxygen). Examples of heteroaromatic functionalities include pyrrole, pyridine, furan, thiophene, triazine, and pterin. Host molecules preferably have more than one aromatic or heteroaromatic functional group (more preferably, at least one aromatic functional group and at least one heteroaromatic functional group).

The carboxy groups can be directly attached to an aromatic or heteroaromatic functional group (for example, carboxyphenyl). For example, when the host molecule has more than one aromatic or heteroaromatic functional group, the carboxy groups can be arranged such that each aromatic or heteroaromatic group has no more than one carboxy group directly attached. Preferably, however, the carboxy groups are not directly attached to an aromatic or heteroaromatic functional group (more preferably, at least one (preferably, all) of the carboxy groups is directly attached to an intervening aliphatic moiety; most preferably, at least one (preferably, all) of the carboxy groups is directly attached to an intervening aliphatic moiety such that at least three covalent bonds separate the carboxy group from an aromatic or heteroaromatic functional group).

The host molecule can be neutral in charge, can have at least one formal positive or negative charge, or can be zwitterionic (that is, carrying at least one formal positive and at least one formal negative charge). Negative charge can be carried, for example, through a carboxy group having a dissociated hydrogen atom, —COO⁻. The negative charge can be shared among multiple carboxy functional groups, such that a proper representation of the host molecule consists of two or more resonance structures. Alternatively, the negative or partial negative charges can be carried by other acid groups in the host molecule. Preferably, the host molecule has a net negative charge of one to four (more preferably, one to two).

Useful host molecules include those that comprise at least one pterin or 5-substituted pterin moiety (especially, pterin). Preferably, the host molecule comprises at least one pteroyl or 5-substituted pteroyl moiety (especially, pteroyl). More preferably, the host molecule is a pteroylglutamic acid (for example, folic acid) or a 5-substituted pteroylglutamic acid (for example, folinic acid). Most preferably, the host molecule is a pteroylglutamic acid, of which folic acid is especially preferred.

Such useful host molecules can be synthesized using known organic chemical techniques, and the pteroylglutamic acids also can be isolated from various food sources (for example, spinach). Folic acid belongs to the group of B-vitamins and is commercially available.

Useful host molecules can generally be capable of forming a chromonic liquid crystal phase or assembly when dissolved in an aqueous solution or an alkaline aqueous solution prior to the addition of multi-valent cations (that is, prior to crosslinking). Chromonic phases or assemblies are well known (see, for example, Handbook of Liquid Crystals, Volume 2B, Chapter XVIII, Chromonics, John Lydon, pp. 981-1007, Wiley-VCH, New York (1998)) and generally consist of stacks of flat, multi-ring aromatic or heteroaromatic molecules. The molecules generally consist of a hydrophobic core surrounded by hydrophilic groups. The stacking can assume a number of different morphologies, but is typically characterized by a tendency to form columns created by a stack of layers. Ordered stacks of molecules are formed that grow with increasing concentration, but that are distinct from micellar phases in that they generally do not have surfactant-like properties and do not exhibit a critical micellar concentration. Typically, the chromonic phases will exhibit isodesmic behavior (that is, addition of molecules to the ordered stack leads to a monotonic decrease in free energy).

Useful host molecules include those that can form a chromonic M, N, or isotropic phase in aqueous solution or alkaline aqueous solution before they are in the presence of multi-valent cations (that is, before crosslinking). The chromonic M phase typically is characterized by ordered stacks of molecules arranged in a hexagonal lattice. The chromonic N phase is characterized by a nematic array of columns (that is, there is long range ordering along the columns characteristic of a nematic phase, but there is little or no ordering amongst the columns, making the phase less ordered than an M phase). The chromonic N phase typically exhibits a schlieren texture, which is characterized by regions of varying index of refraction in a transparent medium.

### Water-Insoluble Matrix

The water-insoluble matrix of the composition of the invention comprises host molecules that are non-covalently crosslinked by multi-valent cations. This crosslinking forms a three-dimensional matrix that is insoluble in water. As used herein, the term “non-covalent” means that the crosslinking bond can be formed and cleaved reversibly in the presence of a solvent. That is, the crosslinking results from associations of the cations with the host molecules that are strong enough to hold the molecules together (for example, through ionic bonding or coordinate covalent bonding).

These associations can result from interaction of a formal negative charge on the host molecule with the formal positive charge of a multi-valent cation. Since the multi-valent cation has at least two positive charges, it is able to form an association (for example, an ionic bond) with two or more host molecules (that is, a crosslink between two or more host molecules). The crosslinked, water-insoluble matrix arises from the combination of direct host molecule-host molecule interactions (for example, π-π interactions) and host molecule-cation interactions.

Cations having a charge of at least about 2 can be used, but divalent and/or trivalent cations are generally preferred. It can be more preferred that a majority of the multi-valent cations are divalent. Suitable cations include any divalent or trivalent cations, with calcium, magnesium, zinc, aluminum, and iron being particularly preferred. Mixtures of different cations can be used if desired.

As described above, a chromonic phase or assembly of the host molecules in aqueous solution can comprise columns created from layered stacks of individual host molecules or layered stacks of associations of host molecules (for example, lateral associations such as Hoogsteen-type hydrogen-bonded folate tetramers). The multi-valent cations can provide crosslinks between these columns. Although not wishing to be bound by any particular theory, it is believed that the host molecules also can associate with each other through, for example, interaction of the aromatic or heteroaromatic functionality and the carboxy functionality. Alternatively, a multi-valent cation can associate with two or more host molecules. For example, a divalent cation can form a “dimer” that can become insoluble, and the insoluble “dimers” can interact with each other through the host molecule functionality to form a water-insoluble matrix.

As used herein in reference to a matrix, “water-insoluble” means that the matrix is essentially insoluble in substantially pure water (for example, deionized or distilled water), having a solubility of less than about 0.01 weight percent at 25° C. In some embodiments, the matrix can be in the form of a fine particulate that can be suspended and/or uniformly dispersed within an aqueous solution, but such dispersibility is not to be equated with solubility.

In some cases an aqueous solution can contain free host molecules and/or free multi-valent cations that are soluble in the aqueous solution when present as isolated, or free, molecules. Such free host molecules and/or free multi-valent cations, however, are not in the form of a water-insoluble matrix of the composition of the invention. Under certain conditions, a water-insoluble matrix will dissolve in cation-containing aqueous solutions, as will be evident from the description below on release of guest molecules, but such dissolution in specific cation-containing aqueous solutions is not indicative of water solubility.

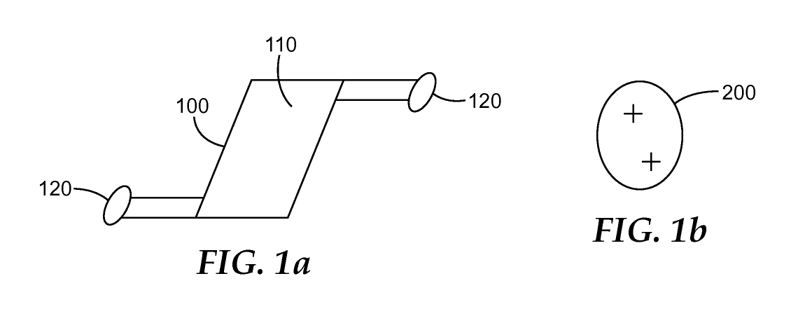

The water-insoluble matrices can be capable of encapsulating a guest molecule and subsequently controllably releasing the guest molecule. Although numerous morphologies can arise depending on the particular chemical natures and amounts of the host molecules and multi-valent cations, a schematic representation of embodiments of such a matrix and its components is set forth in FIGS. 1-4.

FIGS. 1a and 1b show an isolated host molecule or host molecule association 100 and an isolated multi-valent cation 200. The host molecule or host molecule association 100 has aromatic or heteroaromatic functionality 110 that is schematically represented as a planar or sheet-like area within the host molecule or host molecule association 100. The host molecule or host molecule association 100 also has at least two carboxy functional groups 120 that are indirectly attached (for example, by being directly bonded to intervening aliphatic moieties) to the aromatic or heteroaromatic functionality 110. The multi-valent cation 200 is schematically represented by an oval.

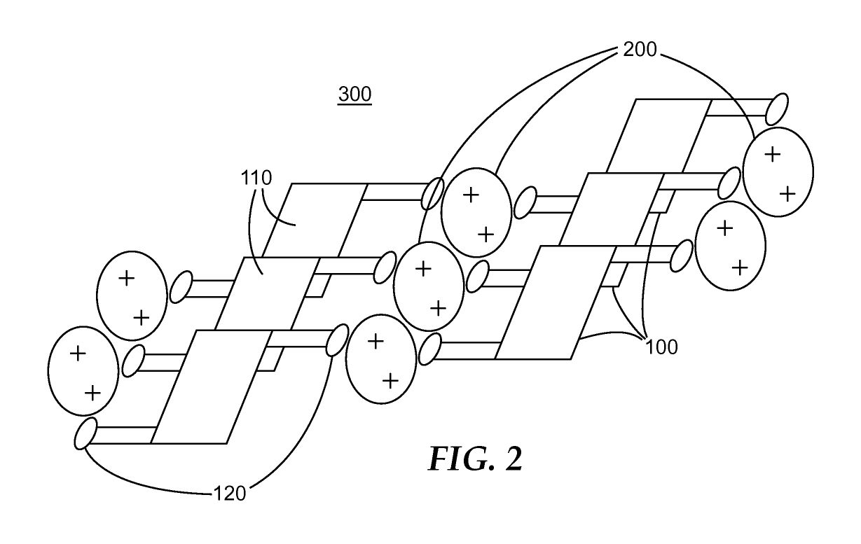

FIG. 2 shows one embodiment of a water-insoluble matrix 300. The aromatic or heteroaromatic functionalities 110 of adjacent host molecules or host molecule associations 100 align to form a layered stack of host molecules or host molecule associations. These layered stacks have additional interactions between their carboxy groups 120 and the multi-valent cations 200, which provides for crosslinking between the layered stacks because of the multiple valency of the cations. As shown in FIG. 2, a divalent cation creates a non-covalent, bridging linkage between carboxy groups 120 on two different host molecules or host molecule associations 100. Although not shown, additional valency of a cation would allow for additional non-covalent, bridging linkages between carboxy groups 120.

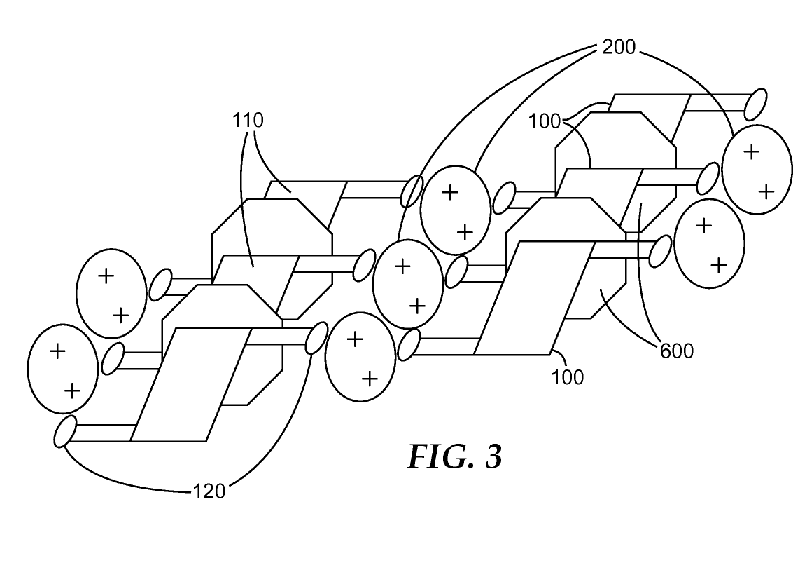

The water-insoluble matrices of the composition of the invention can further comprise a guest molecule that can be encapsulated within the matrix and subsequently released. Encapsulation of a guest molecule 600 is represented schematically in FIG. 3, where a guest molecule 600 is encapsulated between a pair of host molecules or host molecule associations 100. Although FIG. 3 shows an individual interleaving of guest and host molecules or host molecule associations, it should be understood that encapsulation can occur in a variety of ways and thus is to be more broadly interpreted.

The guest molecule can be dispersed within the matrix such that it is encapsulated, and, as such, the guest molecule can be effectively isolated by the matrix from an outside environment. For example, a guest molecule that is ordinarily soluble in water can be prevented from dissolving in water by encapsulation within the water-insoluble matrix. Similarly, guest molecules that are unstable in the presence of an acid can be effectively isolated by the matrix so that they do not significantly degrade.

In the embodiment of FIG. 3, guest molecules 600 are individually intercalated in the matrix 300. That is, the guest molecules are present within the matrix as isolated molecules surrounded by the host molecules or host molecule associations, rather than as aggregates of guest molecules dispersed within the matrix. When the guest and host molecules have similar dimensions, intercalation can take the form of an alternating structure of host and guest molecules. When a guest molecule is substantially larger than a host molecule, several host molecules (for example, that constitute a host molecule association) or several host molecule associations or even several host molecule stacks can surround a single guest molecule. Conversely, when a guest molecule is substantially smaller than a host molecule, more than one guest molecule can be encapsulated between adjacent host molecules. Mixtures of more than one type of guest molecule can be encapsulated within a single matrix.

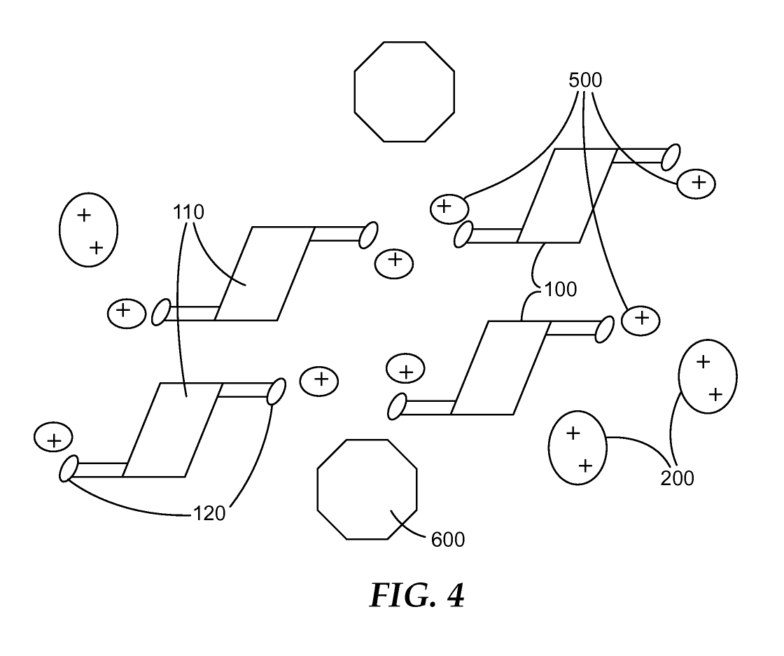

Referring to FIG. 4, if the multi-valent cations 200 are, for example, replaced by univalent cations 500 in an aqueous solution, then the non-covalent, bridging linkages can be reversibly cleaved. The univalent cations will tend to associate only with a single carboxy group 120, and this can allow the host molecules or host molecule associations 100 to dissociate from each other and release the guest molecules 600. Release of a guest molecule will depend on a number of factors, including the types and amounts of guest molecules, the types and amounts of multi-valent cations present, the types and amounts of host molecules, and the environment into which the matrix is placed.

FIGS. 1-4 and the above description are intended to illustrate the general nature of the composition of the invention. Thus, it should be understood that the depictions are not intended to specify precise bonding interactions or detailed three-dimensional structure, and these schematics should not be considered to be limiting to the scope of the invention. Rather, the description below provides additional explanation of the constituent components of the composition of the invention and their arrangement.

### Guest Molecules

The composition of the invention can be used to encapsulate and release a guest molecule. Examples of useful guest molecules include dyes, cosmetic agents, fragrances, flavoring agents, and bioactive compounds (for example, drugs, herbicides, pesticides, pheromones, and antifungal agents). As used herein, a bioactive compound is a compound that can be used in the diagnosis, cure, mitigation, treatment, or prevention of disease, or that can be used to affect the structure or function of a living organism. Drugs (that is, pharmaceutically active ingredients) are particularly useful guest molecules that are intended to have a therapeutic effect on an organism. Herbicides and pesticides are examples of bioactive compounds intended to have a negative effect on a living organism (for example, a plant or pest).

Although essentially any type of drug can be employed in the composition of the invention, particularly suitable drugs include those that are relatively unstable when formulated as solid dosage forms, those that are adversely affected by the low pH conditions of the stomach, those that are adversely affected by exposure to enzymes in the gastrointestinal tract, and those that are desirable to provide to a patient via sustained or controlled release. Examples of suitable drugs include antiinflammatory drugs, both steroidal (for example, hydrocortisone, prednisolone, and triamcinolone) and nonsteroidal (for example, naproxen and piroxicam); systemic antibacterials (for example, erythromycin, tetracycline, gentamycin, sulfathiazole, nitrofurantoin, vancomycin, penicillins such as penicillin V, cephalosporins such as cephalexin, and quinolones such as norfloxacin, flumequine, ciprofloxacin, and ibafloxacin); antiprotazoals (for example, metronidazole); antifungals (for example, nystatin); coronary vasodilators; calcium channel blockers (for example, nifedipine and diltiazem); bronchodilators (for example, theophylline, pirbuterol, salmeterol, and isoproterenol); enzyme inhibitors such as collagenase inhibitors, protease inhibitors, elastase inhibitors, lipoxygenase inhibitors, and angiotensin converting enzyme inhibitors (for example, captopril and lisinopril); other antihypertensives (for example, propranolol); leukotriene antagonists; anti-ulceratives such as H2 antagonists; steroidal hormones (for example, progesterone, testosterone, and estradiol); local anesthetics (for example, lidocaine, benzocaine, and propofol); cardiotonics (for example, digitalis and digoxin); antitussives (for example, codeine and dextromethorphan); antihistamines (for example, diphenhydramine, chlorpheniramine, and terfenadine); narcotic analgesics (for example, morphine and fentanyl); peptide hormones (for example, human or animal growth hormones, luteinizing hormone-releasing hormone (LH-RH)); cardioactive products such as atriopeptides; proteinaceous products (for example, insulin); enzymes (for example, anti-plaque enzymes, lysozyme, and dextranase); antinauseants; anticonvulsants (for example, carbamazine); immunosuppressives (for example, cyclosporine); psychotherapeutics (for example, diazepam); sedatives (for example, phenobarbital); anticoagulants (for example, heparin); analgesics (for example, acetaminophen); antimigraine agents (for example, ergotamine, melatonin, and sumatripan); antiarrhythmic agents (for example, flecamide); antiemetics (for example, metoclopromide and ondansetron); anticancer agents (for example, methotrexate); neurologic agents such as anti-depressants (for example, fluoxetine) and anti-anxiolytic drugs (for example, paroxetine); hemostatics; and the like; as well as pharmaceutically acceptable salts and esters thereof.

Proteins and peptides can be particularly suitable for use in the composition of the invention. Suitable examples include erythropoietins, interferons, insulin, monoclonal antibodies, blood factors, colony stimulating factors, growth hormones, interleukins, growth factors, therapeutic vaccines, and prophylactic vaccines. The amount of drug that constitutes a therapeutically effective amount can be readily determined by those skilled in the art with due consideration of the particular drug, the particular carrier, the particular dosing regimen, and the desired therapeutic effect. The amount of drug will typically vary from about 0.1 to about 70 percent by weight of the total weight of the water-insoluble matrix. The drug can be, for example, intercalated in the matrix.

A preferred drug is insulin. Insulin is a polypeptide hormone that regulates carbohydrate metabolism. Multiple, daily subcutaneous injections of insulin can often be necessary to regulate the blood sugar of humans with diabetic conditions. Orally administered insulin would be highly desirable to improve patient compliance and convenience, as well as to provide the therapeutic benefits of insulin to patients with borderline diabetic conditions without the need for injection training and compliance. Without some form of protection or encapsulation, however, orally administered insulin would be digested in the stomach by the same mechanism as for other proteins.

In addition to the above-described drugs, the guest molecule can be an antigen for use as a vaccine, or it can be an immune response modifier (IRM) compound. If desired, both an antigen and an immune response modifier can be present as guest molecules in a single matrix, and the immune response modifier compound can act, for example, as a vaccine adjuvant by activating toll-like receptors. Examples of immune response modifier compounds include molecules known to induce the release of cytokines (such as, for example, Type I interferons, TNF-α, IL-1, IL-6, IL-8, IL-10, IL-12, IP-10, MIP-1, MIP-3, and/or MCP-1) and also to inhibit production and secretion of certain TH-2 cytokines (such as IL-4 and IL-5). Combined delivery of an immune response modifier and an antigen can elicit an enhanced cellular immune response (for example, cytotoxic T lymphocyte activation) and a switch from a Th2 to Th1 immune response.

The IRM compound(s) used as guest molecules can be small molecule IRMs, which are relatively small organic compounds (for example, having a molecular weight less than about 1000 daltons, preferably less than about 500 daltons), or larger biologic molecule IRMs (for example, oligonucleotides such as cytosine-guanine dinucleotides (CpG)). Combinations of such compounds can also be used. Suitable small molecule IRMs include compounds comprising a 2-aminopyridine fused to a five-membered nitrogen-containing heterocyclic ring such as, for example, imidazoquinolin-4-amines (for example, imiquimod and resiquimod), imidazonaphthyridin-4-amines (for example, compounds described in U.S. Pat. No. 6,194,425 (Gerster)), imidazopyridin-4-amines (for example, compounds described in U.S. Pat. No. 5,446,153 (Lindstrom)), thiazoloquinolin-4-amines (for example, compounds described in U.S. Pat. No. 6,110,929 (Gerster)), and pyrazoloquinolin-4-amines (for example, compounds described in International Publication No. 2005/079195 (Hays)).

### Preparation of Composition

In one aspect, this invention provides a method for preparing a composition for encapsulation and controlled release. The method comprises combining a dispersion (preferably, a dispersion in water or in a mixture of water and organic solvent such as, for example, methanol) of host molecule(s) (and, optionally, guest molecule(s)) with at least one base (for example, at least about one mole of base per mole of host molecule up to about one mole of base per mole of carboxy functional group) to form a solution having a chromonic phase, and combining the solution having a chromonic phase with a solution of multi-valent ions to form an insoluble composition for drug delivery.

If desired, a guest molecule, such as a drug, can be dissolved in an aqueous surfactant-containing solution prior to introduction of the host molecule. Suitable surfactants include, for example, long chain saturated fatty acids or alcohols and mono- or poly-unsaturated fatty acids or alcohols. Oleic acid is an example of a suitable surfactant. The surfactant can aid, for example, in dispersing the guest molecule so that it can be better encapsulated.

If desired, a base can be added to the guest molecule solution prior to introduction of the host molecule. Alternatively, a base can be added to a host molecule solution prior to adding the guest molecule. Examples of suitable bases include ethanolamine, sodium or potassium hydroxide, amines (mono-, di-, tri-, and polyamines), and the like, and mixtures thereof. Such bases can aid, for example, in dissolving the host compound and in forming a liquid crystalline phase.

Alternatively, the composition of the invention can be prepared as films, coatings, or depots directly in contact with a patient. For example, the multi-valent cations and the host molecule can be mixed together or applied consecutively to a particular site on a patient to form either a coating or a depot at the site, depending upon the method of application. One example of this is to form a topical coating by independently applying the multi-valent cations and the host molecule to the skin of a patient and then allowing them to remain in contact for sufficient time to form a crosslinked matrix. Another example is to independently inject multi-valent cations and host molecules into a body tissue or organ, such as a cancerous tumor, and allow them to remain in contact for sufficient time to form a crosslinked matrix. Yet another example is to independently apply the multi-valent cations and the host molecules directly to an internal tissue during a surgical procedure, for example, to form a crosslinked matrix comprising an antibiotic to reduce the chance of infection after the procedure.

The composition of the invention can optionally include one or more additives such as, for example, initiators, fillers, plasticizers, crosslinkers, tackifiers, binders, antioxidants, stabilizers, surfactants, solubilizers, permeation enhancers, adhesives, viscosity enhancing agents, coloring agents, flavoring agents, and the like, and mixtures thereof.

### Particulate Composition and Medicinal Suspension

In one aspect, the invention provides a particulate composition comprising particles comprising the above-described water-insoluble matrix. A guest molecule can be encapsulated within the matrix and subsequently released. The appropriate size and shape of the particles can vary depending upon their intended use. For example, when a drug is encapsulated within the matrix, the appropriate size and shape of the particles will vary depending upon the type and amount of drug dispersed within the matrix, the intended route of delivery of the particles, and the desired therapeutic effect.

Although large particles (for example, on the order of several millimeters in diameter) can be prepared, the mass median diameter of particles of the particulate composition of the invention can typically be less than about 100 μm in size, usually less than about 25 μm in size, and in some cases less than about 10 μm in size. In certain cases, it can be desired to have particles less than about 1 μm in size. Particles are typically substantially spherical in their general shape but can also take any other suitable shape (for example, needles, cylinders, or plates).

The particles can be prepared by mixing host molecules with multi-valent cations. Typically this can be done by dissolving the host molecule in an aqueous solution (preferably, in an amount of about 5 to about 60 weight percent of host molecule to water), adding base as described above, and subsequently adding multi-valent cations to cause insolubility of the particles, or alternatively, by adding an aqueous solution of dissolved host molecules to a solution of multi-valent cations. Drugs (or other guest molecules) can be dispersed or intercalated in the matrix by adding drug to either the aqueous solution of host molecules or the multi-valent cation solution prior to combining the two solutions. Alternatively, a drug can be dispersed or dissolved in another excipient or vehicle, such as an oil or propellant, prior to mixing with the host molecule or multi-valent cation solution. Particles can be collected by, for example, filtration, spraying, or other means, and then dried to remove the aqueous carrier.

The particles can be dissolved in an aqueous solution of univalent cations or non-ionic compounds (for example, surfactants). Typical univalent cations include sodium and potassium. The concentration of univalent cations needed to dissolve the particles will depend on the type and amount of the host molecules within the matrix, but for complete dissolution of the particles there can generally be at least a molar amount of univalent cations equivalent to the molar amount of carboxy groups in the matrix. In this way, there can be at least one univalent cation to associate with each carboxy group.

The rate at which a particle dissolves can also be varied by adjusting the type and amount of multi-valent cation used for crosslinking. Although divalent cations can be sufficient to crosslink the matrix, higher valency cations can provide additional crosslinking and lead to slower dissolution rates. In addition to valency, dissolution rate can also depend on the particular cation type.

For example, a non-coordinating divalent cation, such as magnesium, can generally lead to faster dissolution than a coordinating divalent cation, such as calcium or zinc. Different cation types can be mixed, so as to give an average cation valency that is not an integer. In particular, a mixture of divalent and trivalent cations can exhibit a slower dissolution rate than a like matrix where all of the cations are divalent.

Often it can be desirable to have all of the guest molecules released over time, but it can be desired in certain applications to have only a portion of the guest molecules released. For example, the type and/or amount of host molecule and/or multivalent cation can be adjusted such that the total amount of guest molecules that are released will vary depending upon the environment into which they are placed. In certain embodiments, the particles cannot dissolve in an acidic solution or in an acidic solution containing univalent cations, thereby protecting acid sensitive guest molecules from degradation.

When the guest molecule is a drug, two common types of general release profiles are immediate release and sustained release. For immediate release, it typically can be desired that most of the drug will be released in a time period of less than about 4 hours, generally less than about 1 hour, often less than about 30 minutes, and in some cases less than about 10 minutes. In some cases, it can even be desirable to have drug release be nearly instantaneous (for example, occurring in a matter of seconds).

For sustained (or controlled) release, it typically can be desired that most of the drug will be released over a time period greater than or equal to about 2 hours. Periods of one month or more can be desired, for example in various implantable applications. Oral sustained release dosages can generally release most of the drug over a time period of about 4 hours to about 14 days, sometimes about 12 hours to about 7 days. It can be desirable, however, to release most of the drug over a time period of about 24 to about 48 hours. A combination of immediate and sustained release can also be desirable, where, for example, a dosage can provide an initial burst of release to rapidly alleviate a particular condition, followed by a sustained delivery to provide extended treatment of the condition.

In some cases, it can be desirable to have a pulsatile or multi-modal release of drug, such that the rate of release varies over time (for example, increasing and decreasing to match the circadian rhythm of an organism). Similarly, it can be desirable to provide a delayed release of drug, such that a dosage can be administered at a convenient time (such as just before going to sleep), but release of the drug can be prevented until a later time when it may be more efficacious (such as just before waking). One approach for achieving pulsatile, multi-modal, or delayed release profiles can be to mix two or more types of particles having different drug release characteristics. Alternatively, particles can be formed having two or more distinct phases, such as a core and a shell, having different drug release characteristics.

In a further aspect, this invention provides a medicinal suspension formulation comprising the particulate composition of the invention and a liquid (for example, at least one liquid, pharmaceutically acceptable carrier).

### Drug Delivery Processes

The particulate composition of the invention can be particularly useful in oral dosage drug delivery. Typical oral dosage forms include solid dosages (such as tablets and capsules) and other dosages administered orally (such as liquid suspensions and syrups). When administered to an animal, some embodiments of the particles can be stable in the acidic environment of the stomach and then dissolve when passed into the non-acidic environment of the intestine. When the particles are stable in acidic solution, the particles can generally be stable for periods of time longer than about 1 hour, sometimes for more than about 12 hours, and sometimes for more than about 24 hours, when present in an acidic environment with a pH less than 7.0 (for example, less than about 5.0, and in some cases less than about 3.0).

In certain embodiments of the particulate composition of the invention, the mass median aerodynamic diameter of drug-containing particles can be often less than about 10 μm and in some cases less than about 5 μm, such that the particles are respirable when delivered to the respiratory tract of an animal via an inhalation route of delivery. Delivery of particles by inhalation is well known and can be accomplished by various devices, including pressurized meter dose inhalers (for example, those described in U.S. Pat. No. 5,836,299 (Kwon, et al.), the description of which is incorporated herein by reference); dry powder inhalers (for example, those described in U.S. Pat. No. 5,301,666 (Lerk et al.), the description of which is incorporated herein by reference); and nebulizers (for example, those described in U.S. Pat. No. 6,338,443 (Piper, et al.), the description of which is incorporated herein by reference). Respirable particles of the particulate composition of the invention can be incorporated into an inhalation dosage form using known methods and processes.

Drug-containing particles of the particulate composition of the invention can be delivered by routes other than orally or by inhalation. For example, the particles can be delivered by intravenous, intramuscular, or intraperitoneal injection (for example, in the form of aqueous or oil solutions or suspensions); by subcutaneous injection; and by incorporation into transdermal, topical, and mucosal dosage forms (for example, creams, gels, adhesive patches, suppositories, and nasal sprays). The particulate composition can also be implanted or injected into various internal organs and tissues (for example, cancerous tumors) or can be directly applied to internal body cavities (for example, during surgical procedures).

Particle suspensions in propellants, such as hydrofluorocarbons or other suitable propellants, can find use in pressurized meter dose inhalers used for inhalation or nasal drug delivery. Particle suspensions in aqueous-based media can find use in nebulizers used for inhalation or nasal drug delivery. Alternatively, particle suspensions in aqueous media can also find utility in intravenous or intramuscular delivery.

Thus, in at least one aspect, the invention provides method(s) for drug delivery to an organism (for example, a plant or animal). One method comprises (a) providing the composition of the invention comprising an encapsulated drug; (b) delivering the composition to an organism such that it comes into contact with a composition comprising univalent cations and releases at least a portion of the encapsulated drug; and (c) allowing the released drug to remain in contact with at least a part of the organism for a period of time sufficient to achieve a desired therapeutic effect.

In some embodiments of this method, the composition can be delivered to an animal orally, and, in some such embodiments, the composition cannot release the encapsulated drug until it has passed into the intestine. The encapsulated drug can be released immediately upon passing into the intestine, or it can be released in a sustained fashion while residing within the intestine. The encapsulated drug can also pass into or across the intestinal membrane and release the drug elsewhere in the animal (for example, in the circulatory system). In still other embodiments, the composition can be delivered via oral or nasal inhalation.

### EXAMPLES

Objects and advantages of this invention are further illustrated by the following examples, but the particular materials and amounts thereof recited in these examples, as well as other conditions and details, should not be construed to unduly limit this invention. These examples are merely for illustrative purposes only and are not meant to be limiting on the scope of the appended claims.

All parts, percentages, ratios, etc. in the examples and the rest of the specification are by weight, unless noted otherwise. Solvents and other reagents used were obtained from Sigma-Aldrich Chemical Company, St. Louis, Mo., or Alfa Aesar of Ward Hill, Mass., unless otherwise noted. Ringer's solution, a balanced aqueous solution of 600 mg NaCl (USP Grade), 310 mg sodium lactate, 30 mg KCl (USP Grade), and 20 mg CaCl₂ (USP Grade) per 100 mL having pH 6.5 (6.0-7.5) used in physiological experiments, was obtained from Baxter of Deerfield, Ill. as “Lactated Ringer's Injection USP.”

### Determination of Insulin Concentrations

Insulin concentrations were determined using high performance liquid chromatography (HPLC) using a reversed-phase gradient elution technique. A 150×4.6 mm Zorbax Stablebond C8 (SB-C, Agilent Technologies, Wilmington, Del.) silica column was equilibrated with a 85/15 volume to volume (v/v) mixture of water and acetonitrile containing 0.1 percent by volume of trifluoroacetic acid at 1.0 mL/minute and 25° C. Following a 15 μL injection of a sample solution, insulin was eluted with a 10 minute linear gradient to a 30/70 v/v mixture of water and acetonitrile containing 0.1 percent by volume of trifluoroacetic acid. The elution of insulin was detected using ultraviolet absorbance detection at 210 nm. The peak area measured in this experiment was compared with the response of standard solutions of bovine insulin analyzed under the same conditions in order to determine the concentration of insulin in the sample solution.

### Preparation of 3-{4,6-Bis[(4-carboxyphenyl)amino-1,3,5-triazin-2-yl}-1-methyl-1H-imidazol-3-ium Zwitterion Hydrate (hereinafter, “Comparative Compound ZH”)

3-{4,6-bis[(4-carboxyphenyl)amino-1,3,5-triazin-2-yl}-1-methyl-1H-imidazol-3-ium chloride (hereinafter, “Comparative Compound,” corresponding to the lefthand structure below) (prepared essentially by the method described in Example 1 of U.S. Pat. No. 6,488,866 (Sahouani et al.), except using 1-methylimidazole instead of 4-N,N-dimethylaminopyridine; 78.68 g; 65 weight percent purity as determined by base titration) was added to deionized water (450 mL) while stirring, and the resulting mixture was then mixed for 30 minutes before addition of base. Sodium hydroxide (5.27 mL, 50 weight percent in water) was added drop-wise to the mixture over a period of 15 minutes. The resulting solid-liquid mixture was then mixed for an additional 90 minutes. A product (corresponding to the central structure below) formed after storage for an additional 2-3 hours, and the product was filtered and air dried.

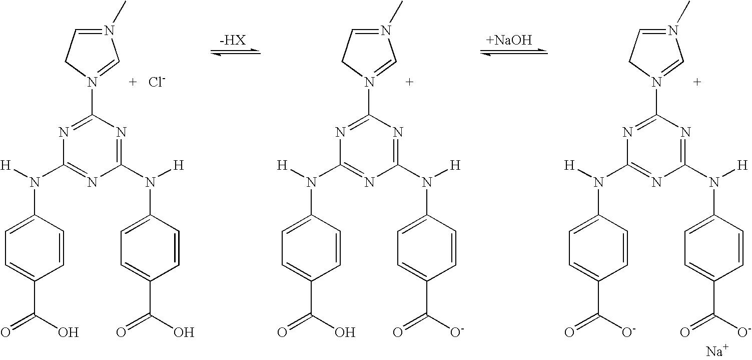

### Titrations

### Comparative Titration

Titration of 0.5 Weight Percent Comparative Compound with More than 3 Equivalents of Base

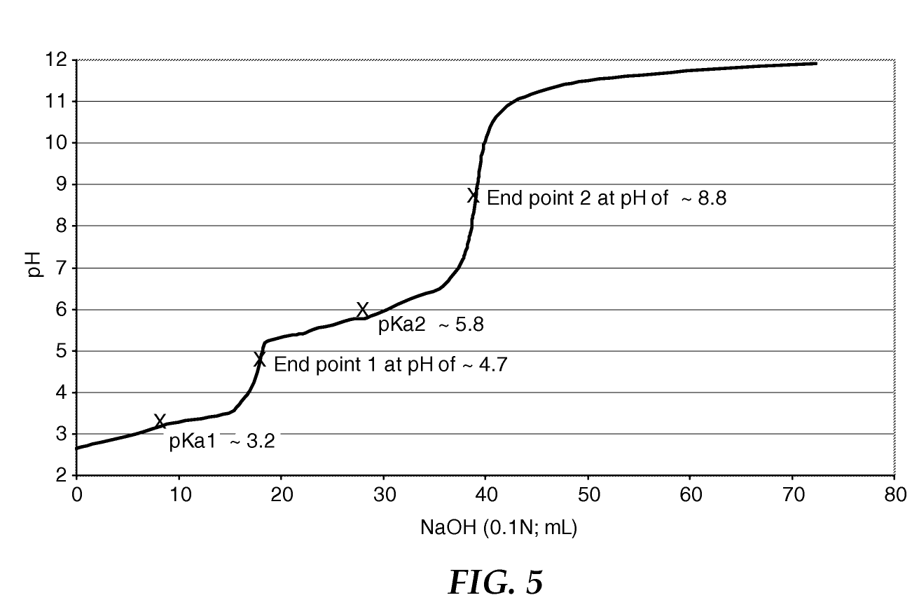

The Comparative Compound (1.0 g) was dispersed in deionized water (199 mL) using a shear mixer/emulsifier (Silverson Model L4R, Silverson Machines, Ltd., Waterside, Chesham, Bucks, England) for approximately 5 minutes. Cresol red indicator (0.04 weight percent in water; 4 drops) was added to aid in end point detection. Samples were titrated (using a 50 mL buret with 0.1 mL graduation) with rapid stirring to maintain suspension of dispersed solid in the liquid medium (0.1 N analytical standard grade sodium hydroxide from Mallinckrodt Baker, Phillipsburg, N.J.) over a period of 1-3 hours. The dispersion became clear after 2 equivalents of base had been added, indicating conversion of the Comparative Compound to a compound corresponding to the righthand structure above. As shown in the resulting titration curve of FIG. 5, a first pKa was observed at a pH of about 3.2, and a second pKa was observed at a pH of about 5.8. A first end point was observed at a pH of about 4.7, and a second end point was observed at a pH of about 8.8. At higher concentrations, a liquid crystalline phase potentially useful for encapsulation of guest molecules is formed between a pH of about 7 and a pH of about 8. This phase forms at a steep end point transition of the titration curve, where even a small addition of base caused a significant change in pH (indicating a need for careful monitoring and control of the amount of base addition for use of the Comparative Compound in, for example, the encapsulation of pH-sensitive guest molecules).

### Titration A

Titration of 0.5 Weight Percent Folic Acid with More than 3 Equivalents of Base

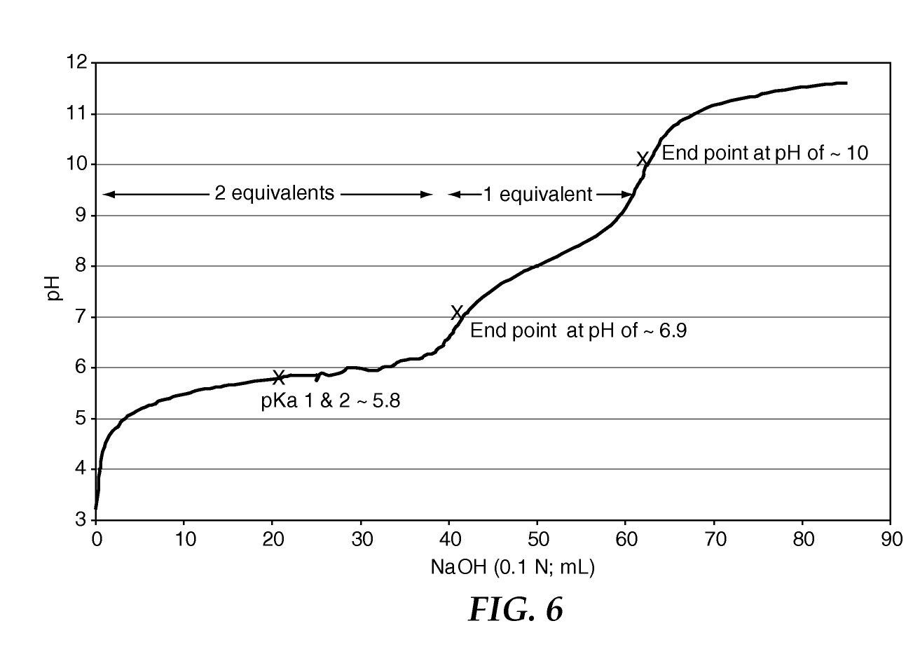

Folic acid (1.0 g) was dispersed in deionized water (199 mL) using a Silverson L4R shear mixer/emulsifier for approximately 5 minutes. Cresol red indicator (0.04 weight percent in water; 4 drops) was added to aid in end point detection. Samples were titrated (using a 50 mL buret with 0.1 mL graduation) with rapid stirring to maintain suspension of dispersed solid in the liquid medium during the addition of base (0.1 N analytical standard grade sodium hydroxide from Mallinckrodt Baker, Phillipsburg, N.J.). The dispersion became clear after 2 equivalents of base had been added. As shown in the resulting titration curve of FIG. 6, a first and a second pKa were observed at a pH of about 5.8. A combined end point was observed at a pH of about 6.9, and a third end point was observed at a pH of about 10.0. A “buffer region” of the titration curve was observed between a pH of about 5.5 and a pH of about 6.5, in which the addition of base did not effect a significant variation in pH and, at higher concentrations, a liquid crystalline phase is achieved without the need for careful monitoring and control of the amount of base added (even for use in, for example, the encapsulation of pH-sensitive guest molecules).

### Titration B

Titration of 1 Weight Percent Folic Acid with More than 3 Equivalents of Base

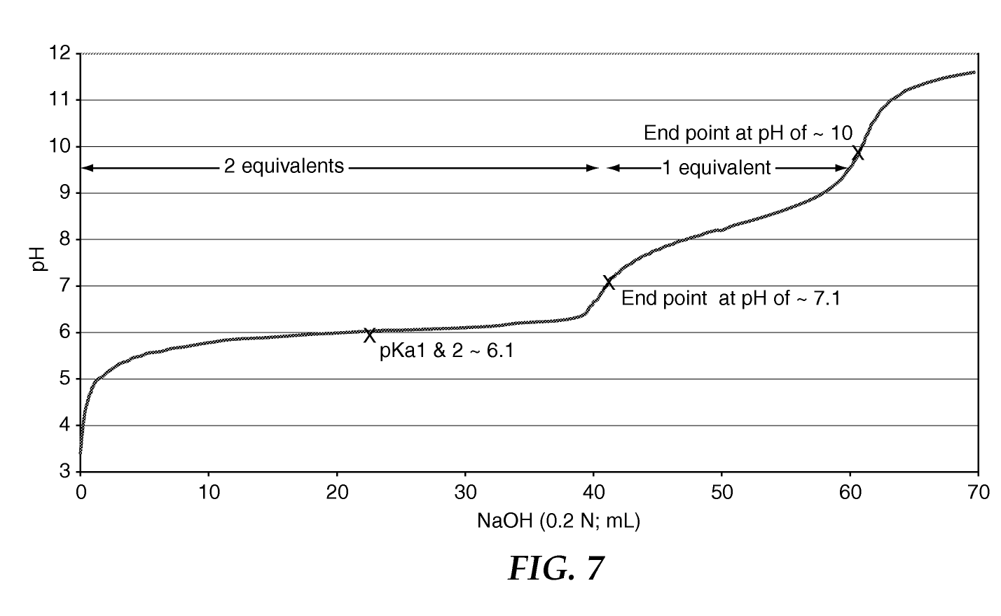

Folic acid (2.0 g) was dispersed in deionized water (198 mL) using a Silverson L4R shear mixer/emulsifier for approximately 5 minutes. Cresol red indicator (0.04 weight percent in water; 4 drops) was added to aid in end point detection. Samples were titrated (using a 50 mL buret with 0.1 mL graduation) with rapid stirring to maintain suspension of dispersed solid in the liquid medium during the addition of base (0.2 N analytical standard grade sodium hydroxide from Mallinckrodt Baker, Phillipsburg, N.J.). The dispersion became clear after 2 equivalents of base had been added. As shown in the resulting titration curve of FIG. 7, a first and a second pKa were observed at a pH of about 6.1. A combined end point was observed at a pH of about 7.1, and a third end point was observed at a pH of about 10.0. A “buffer region” of the titration curve was observed between a pH of about 5.5 and a pH of about 6.5, in which the addition of base did not effect a significant variation in pH and, at higher concentrations, a liquid crystalline phase is achieved without the need for careful monitoring and control of the amount of base added (even for use in, for example, the encapsulation of pH-sensitive guest molecules).

### Comparative Example 1

### Attempted Preparation of Particulate Compositions Using Comparative Compound ZH

Comparative Compound ZH (1.5 g) was dispersed in water (8.5 mL; 15 weight percent in solution). The dispersion was then added drop-wise to a series of sample vials containing calcium chloride, zinc chloride, or a mixture of calcium chloride and zinc:chloride (1:1) in deionized water (25 mL, 10 weight percent solution). When the drops of dispersion contacted the surface of each salt solution, the solids in the drops dispersed to form a white powdery precipitate and a few agglomerated chunks.

The above procedure was repeated with the addition of 0.5 equivalent of base per 1 equivalent of Comparative Compound ZH. To a dispersion of Comparative Compound ZH (1.5 g) in water (8.2 mL) was added sodium hydroxide (0.3 mL, 5 N) (15 weight percent in solution), and the resulting mixture was stirred to obtain a homogeneous mixture. This mixture was then added drop-wise to sample vials containing calcium chloride, zinc chloride, or a mixture of calcium chloride and zinc chloride (1:1) in deionized water (25 mL, 10 weight percent solution). When the drops of dispersion contacted the surface of each salt solution, the solids in the drops dispersed to form beads, but the beads did not maintain their integrity and some flaking on the beads was observed after about 3 to 4 days. Un-neutralized Comparative Compound ZH settled out of solution as a powder. After stirring was discontinued, the resulting mixture appeared to comprise a pearlescent solution (about half the total volume) and the powder.

### Example 1

### Preparation of Particulate Compositions Using Folic Acid

Solutions of folic acid having the concentrations shown in Table 1 below were prepared by dispersing folic acid dihydrate (in the amounts shown in Table 1) in deionized water using a magnetic stirrer and then neutralizing by the addition of one or two equivalents of the bases listed in Table 1 (potassium hydroxide or sodium hydroxide, 1.0 N solutions, analytical standard grade available from Mallinckrodt Baker, Phillipsburg, N.J.; concentrated (28-30 weight percent) ammonium hydroxide) with stirring. The resulting liquid crystalline solutions exhibited varying colors and textures.

The sodium hydroxide-neutralized liquid crystalline solutions (10 weight percent folic acid) were then added drop-wise to a series of solutions of calcium chloride dihydrate, calcium acetate, or calcium nitrate each having a concentration of 10 weight percent in water. When the drops of liquid crystalline solution contacted the surface of each salt solution, the drops retained their shape and solidified to form beads (see also Examples 2-4 below). In contrast with Comparative Example 1, the liquid crystalline folic acid solutions with one equivalent of added base did not separate after stirring was discontinued.

TABLE 1

| | | Folic Acid | Amount | Amount | Amount |
|---|---|---|---|---|---|
| | | Concentration | of Folic | of | of Base |
| | Equivalents | (Weight | Acid | Water | Solution |
| Base | of Base | Percent) | (g) | (mL) | (mL) |
| KOH | 1 | 10 | 0.500 | 3.46 | 1.04 |
| KOH | 2 | 10 | 0.500 | 3.46 | 2.08 |
| NH₄OH | 1 | 10 | 0.500 | 4.43 | 0.07 |
| NH₄OH | 2 | 10 | 0.500 | 4.35 | 0.141 |
| NaOH | 1 | 10 | 0.500 | 3.46 | 1.04 |
| NaOH | 2 | 10 | 0.500 | 3.46 | 2.08 |
| KOH | 1 | 15 | 0.751 | 2.68 | 1.57 |
| KOH | 2 | 15 | 0.751 | 1.11 | 3.14 |
| NH₄OH | 1 | 15 | 0.750 | 4.14 | 0.106 |
| NH₄OH | 2 | 15 | 0.750 | 4.03 | 0.212 |
| KOH | 1 | 20 | 1.00 | 1.91 | 2.09 |
| KOH | 2 | 20 | 1.00 | 0.00 | 4.18 |

### Example 2

### Preparation of Particulate Composition Comprising Folic Acid and Encapsulated Evan's Blue Dye Using One Equivalent of Base

Anhydrous folic Acid (FA, 3.0 g) was dispersed in deionized water (15.1 mL). To this dispersion, while stirring was added sodium hydroxide (1.36 mL, 5 N) drop-wise over 5 minutes to provide a pearlescent, orange solution with 15 weight percent solids. Evan's blue dye ((6,6′-[dimethyl[1,1′-biphenyl]-4,4′-diyl)bis(azo)]bis[4-amino-5-hydroxy-1,3-naphthalene disulfonic acid] tetrasodium salt, EB, 0.015 g) dissolved in water (1.02 mL) was added to the folic acid solution and stirred for about 10 minutes to provide a green pearlescent solution that contained 0.5 weight percent EB based upon the weight of FA. This solution was then added drop-wise to a series of vials containing calcium chloride, zinc chloride, or a mixture of calcium chloride and zinc chloride (1:1) in deionized water (25 mL, 10 weight percent solution). When the drops of EB-containing FA solution contacted the surface of each salt solution, the drops retained their shape and solidified, forming crosslinked FA beads that were nominally round and that did not disperse to form a powder. EB consistently remained in the crosslinked FA beads, as indicated by the absence of blue coloration in each solution above the beads. After three days, some of the beads from each solution were put into deionized water, and the beads retained the EB, as indicated by the absence of blue coloration of the water above the beads.

### Example 3

### Preparation of Particulate Composition Comprising Folic Acid and Encapsulated Evan's Blue Dye Using 1.5 Equivalents of Base

Anhydrous folic Acid (FA, 1.5 g) was dispersed in deionized water (7.5 mL). To this dispersion while stirring was added sodium hydroxide (1.02 mL, 5 N) drop-wise over 5 minutes to provide a pearlescent, orange solution with 15 weight percent solids. Evan's blue dye (EB, 0.0075 g) dissolved in water (0.51 mL) was added to the folic acid solution and stirred for about 10 minutes to provide a cloudy orange solution that contained 0.5 weight percent EB based upon the weight of FA. This solution was then added drop-wise to a series of vials containing calcium chloride, zinc chloride, or a mixture of calcium chloride and zinc chloride (1:1) in deionized water (25 mL, 10 weight percent). When the drops of EB-containing FA solution contacted the surface of each salt solution, the drops retained their shape and solidified, forming crosslinked FA beads that were nominally round and that did not disperse to form a powder. EB consistently remained in the beads, as indicated by the absence of blue coloration in the solution above the beads. After three days, some of the beads from each solution were put into deionized water and the beads retained the EB, as indicated by the absence of blue coloration of the water above the beads.

### Example 4

### Preparation of Particulate Composition Comprising Folic Acid and Encapsulated Evan's Blue Dye Using Two Equivalents of Base

Anhydrous folic Acid (FA, 1.5 g) was dispersed in deionized water (7.15 mL). To this dispersion while stirring was added sodium hydroxide (1.3 mL, 5 N) drop-wise over 5 minutes to provide a pearlescent, orange solution with 15 weight percent solids. Evan's blue dye (EB, 0.0075 g) dissolved in water (0.51 mL) was added to the folic acid solution and stirred for about 10 minutes to provide a cranberry red solution that contained 0.5 weight percent EB based upon the weight of FA. This solution was then added drop-wise to a series of vials containing calcium chloride, zinc chloride, or a mixture of calcium chloride and zinc chloride (1:1) in deionized water (25 mL, 10 weight percent). When the drops of EB-containing FA solution contacted the surface of each salt solution, the drops retained their shape and solidified, forming crosslinked FA beads that were nominally round and that did not disperse to form a powder. EB consistently remained in the beads, as indicated by the absence of blue coloration in the solution above the beads. After three days, some of the beads from each solution were put into deionized water, and the beads retained the EB, as indicated by the absence of blue coloration of the water above the beads.

### Example 5

### Preparation of Particulate Composition Comprising Folic Acid and Encapsulated Insulin

A mixture of folic acid dihydrate (6.67 g, 12 weight percent stock solution neutralized with sodium hydroxide to pH 6.2; about 1 equivalent of base) and bovine insulin (1.33 g of a 75 mg/mL stock solution in water; obtained from Sigma-Aldrich as Catalog No. 15500) were placed in a wide-mouth vial containing a stir bar and stirred for 30 minutes. An emulsion of this folic acid/insulin mixture (7.8 g) in hydroxypropylcellulose (155 g of a 17 weight percent solution in water, MW 100,000) was made using a mixer equipped with a propeller for 1 hour. A portion of this emulsion (38.2 g) was added to a crosslinking solution (200 mL) made of calcium chloride and zinc chloride (10 weight percent stock solution of a 1:1 mixture in water) and allowed to remain undisturbed for 1 hour. The resulting mixture was then placed on a shaker (a “Reciprocating Shaker,” catalog number 6010, Eberback Corp., Ann Arbor, Mich.) for 30 minutes. Additional water (200 mL) was then added to this mixture. After gentle mixing, the mixture was centrifuged at 3000 revolutions per minute (rpm) for 30 minutes (and for an additional 30 minutes if the resulting supernatant was cloudy). After removing the supernatant, a portion of which was saved for analysis, additional water (50 mL) was added to the resulting condensed solids (hereinafter, “pellets”), and the resulting sample was ultrasonically probed (30 percent amplitude, Vibracell VCX 130 ultrasonic probe with a 0.64 cm (¼ inch) probe from Sonics & Materials, Inc., Newton, Conn.) for 30 seconds or until pellets were dispersed. After adding water (200 mL) and gentle mixing, the sample was centrifuged at 3000 rpm for 30 minutes. After removing the supernatant, a portion of which was saved for analysis, ethyl alcohol (50 mL) was added, and the sample was ultrasonically probed (30 percent amplitude) for 30 seconds or until pellets were dispersed. After adding additional ethyl alcohol (200 mL) and gentle mixing, the sample was centrifuged at 3000 rpm for 30 minutes. Supernatant was removed, and the sample was placed in a lyophilization jar and was flash frozen using liquid nitrogen. The pellets were then placed in a freeze dryer under vacuum (pressure less than 700 mTorr) until they were powdery.

A portion of the resulting insulin-containing folic acid particles (5 mg) was added to a separate container, along with 5 mL of Ringer's solution. The resulting mixture was then placed on a shaker (a “Reciprocating Shaker,” catalog number 6010, Eberback Corp., Ann Arbor, Mich.), and samples (0.5 mL portions) were removed for analysis after 5, 15, 30, 45, 60, and 90 minutes. The samples were analyzed for insulin content by HPLC, and the results were as follows: 4.8 weight percent released at 5 minutes, 5.9 weight percent released at 15 minutes, 6.6 weight percent released at 30 minutes, 7.5 weight percent released at 45 minutes, and 8.6 weight percent released at 60 minutes.

The referenced descriptions contained in the patents, patent documents, and publications cited herein are incorporated by reference in their entirety as if each were individually incorporated. Various unforeseeable modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only, with the scope of the invention intended to be limited only by the claims set forth herein as follows:

1. A composition comprising a water-insoluble matrix comprising a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety.

2. The composition of claim 1, wherein the substituent at the number 5 position of said moiety is selected from hydrogen, alkyl, formyl, formimino, alkylidene, and alkylidyne.

3. The composition of claim 1, wherein said host molecule comprises at least one pteroyl or 5-substituted pteroyl moiety.

4. (canceled)

5. The composition of claim 3, wherein said host molecule is a pteroylglutamic acid or a 5-substituted pteroylglutamic acid.

6. (canceled)

7. The composition of claim 5, wherein said host molecule is folic acid or folinic acid.

8. (canceled)

9. (canceled)

10. (canceled)

11. (canceled)

12. (canceled)

13. The composition of claim 1, wherein said composition further comprises at least one guest molecule.

14. (canceled)

15. (canceled)

16. The composition of claim 13, wherein said guest molecule is a drug.

17. The composition of claim 16, wherein said drug is selected from proteins and peptides.

18. The composition of claim 17, wherein said drug is insulin.

19. (canceled)

20. (canceled)

21. The composition of claim 1, wherein said multi-valent cations are selected from divalent and trivalent cations.

22. (canceled)

23. A composition comprising a water-insoluble matrix comprising folic acid that is non-covalently crosslinked by multi-valent cations.

24. The composition of claim 23, wherein said composition further comprises at least one guest molecule.

25. The composition of claim 24, wherein said guest molecule is a drug.

26. The composition of claim 25, wherein said drug is selected from proteins and peptides.

27. The composition of claim 26, wherein said drug is insulin.

28. A particulate composition comprising particles comprising a water-insoluble matrix comprising a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety.

29. (canceled)

30. (canceled)

31. (canceled)

32. (canceled)

33. A process for preparing the composition of claim 1 comprising:
(a) combining
(1) a dispersion comprising at least one host molecule that is non-polymeric, that has more than one carboxy functional group, that has at least partial aromatic or heteroaromatic character, and that comprises at least one pterin or 5-substituted pterin moiety, and
(2) at least one base,
to form a solution having a chromonic phase; and
(b) combining said solution having a chromonic phase with a solution of multi-valent cations to form a water-insoluble matrix.

34. A drug delivery process comprising:
(a) providing a composition comprising a water-insoluble matrix comprising
(1) a host molecule that is non-covalently crosslinked by multi-valent cations, that is non-polymeric, that has more than one carboxy functional group, that has at least partial aromatic or heteroaromatic character, and that comprises at least one pterin or 5-substituted pterin moiety, and
(2) at least one drug encapsulated within said matrix;
(b) delivering said composition to an organism such that it comes into contact with a composition comprising univalent cations and releases at least a portion of said encapsulated drug; and
(c) allowing said released drug to remain in contact with at least a part of said organism for a period of time sufficient to achieve a desired therapeutic effect.

35. (canceled)

36. (canceled)

37. (canceled)

38. (canceled)

39. (canceled)

40. A tablet comprising the composition of claim 1.

41. A capsule comprising the particulate composition of claim 28.
